(19) United States
(12) Patent Application Publication
(10) Pub. No.: US 2020/0197604 A1
(43) Pub. Date: Jun. 25, 2020

(54) INFUSION DEVICE CONTROLLER WITH A SET OF INFUSION REGIMES

(71) Applicant: Roche Diabetes Care, Inc., Indianpolis, IN (US)

(72) Inventor: Kurt Friedli, Mannheim (DE)

(21) Appl. No.: 16/805,353

(22) Filed: Feb. 28, 2020

Related U.S. Application Data
(63) Continuation of application No. PCT/EP2018/072834, filed on Aug. 24, 2018.

(30) Foreign Application Priority Data
Aug. 29, 2017 (EP) 17 188 226.9

(51) Int. Cl. A61M 5/168; G16H 20/17

(52) U.S. Cl. CPC A61M 5/16804; G16H 20/17; A61M 2205/50; A61M 2205/581; A61M 2205/582; A61M 2205/8206

(57) ABSTRACT

Disclosed is an infusion device controller, the infusion device controller being designed to control a drive for metered drug infusion out of a drug reservoir according to a set of infusion regimes. The infusion device controller is designed to control infusion in a series of scheduled infusion events and is further designed to determine, for an infusion event volume that is infused in a given scheduled infusion event, an assignment between volume fractions of the infusion event volume and infusion regimes in an interlaced manner.

### RELATED APPLICATIONS

This application is a continuation of PCT/EP2018/072834, filed Aug. 24, 2018, which claims priority to EP 17 188 226.9, filed Aug. 29, 2017, both of which are incorporated herein by reference in their entirety.

### BACKGROUND

The present disclosure is related to the field of infusion technology, in particular drug infusion, infusion devices and infusion device control units. It particularly relates to the infusion of drug according to a number of infusion regimes.

Infusion devices are well known in the art, for example, in the therapy of Diabetes Mellitus by Continuous Subcutaneous Insulin Infusion (CSII) as well as in pain therapy or cancer therapy and are available from a number of suppliers. Throughout this document, a design that is particularly suited for CSII is generally assumed for exemplary purposes.

Infusion devices that are used for CSII are designed to be carried by a Person with Diabetes (PwD), also referred to as “user,” generally continuously night and day. The devices are designed to be carried concealed from view, e.g., with a belt clip or in a pocket, and/or may be designed to be alternatively carried directly attached to the body via an adhesive pad.

State-of-the art infusion devices as used, e.g., in CSII, comprise an electronics control unit, typically based on one or more microprocessors respectively microcontrollers, referred to as infusion device control unit.

Throughout this document, the expressions “infusion device” refers to a device respectively system with at least the before-described basic functionality. The infusion that is carried out with such devices is in any case a metered infusion of controlled volumes. Throughout this document, the term “respectively” is used in several occasions to mean “in particular” or “more specifically.”

Infusion devices as used, for example, in CSII are designed to infuse liquid drug in particular insulin, according to at least two different types of infusion regimes. First, they are designed to infuse liquid drug substantially continuously according to a typically pre-programmed and time variable basal infusion schedule autonomously, i.e., without requiring particular user interactions or operations. Second, they are designed to infuse larger drug boli on demand, for example, to compensate for the intake of carbohydrates as well as to correct undesired high blood glucose values.

With increasing demands regarding comfort and therapy quality, infusion devices have been developed that allow, in addition to basal and bolus infusion as explained before, infusion according to further more advanced infusion regimes, where drug-infusion is spread respectively distributed over a prolonged time period in a uniform or non-uniform way. Some devices further allow an infusion according to a number of infusion regimes in a superimposed way.

Such superimposed infusion, however, significantly increases the complexity of the infusion device controller and in particular regarding the infusion timing and scheduling.

U.S. Pat. No. 9,180,245 B2 discloses a system and method for administering an infusible fluid with sequential multi-part infusion, wherein the sequential, multi-part, infusion event includes a plurality of discrete infusion events. If a one-time infusion event is available to be administered, the administration of at least a portion of the plurality of discrete infusion events included within the sequential, multi-part, infusion event is delayed and the one-time infusion event is administered.

This approach, however requires a comparatively complex control algorithm. Further, the way actual infusion is carried out does not correspond to what was actually programmed respectively requested by a user, thereby potentially confusing the user.

### SUMMARY

This disclosure improves the state of the art regarding drug infusion according to a number of infusion regimes. Favorably, the before-mentioned drawbacks of the state of the art are avoided fully or partly.

In an aspect, an infusion device controller is taught that controls a drive for metered drug infusion according to a set of infusion regimes. The infusion device controller further controls infusion in a series of scheduled infusion events. The infusion device controller is further designed to determine, for an infusion event volume that is infused in a given scheduled infusion event, an assignment between volume fractions of the infusion event volume and infusion regimes in an interlaced manner.

In a further aspect, the infusion device is designed for drug infusion for an extended time period and is to be carried, in particular extracorporeally carried, by a user for the extended time period and concealed from view. The infusion device includes an infusion device controller according to any embodiment as discussed above and/or further below. The infusion device further includes a drive in operative coupling with the infusion device control unit. The drive is further designed to couple to a drug reservoir for infusing drug out of the drug reservoir.

In a further aspect, disclosed is a method for controlling a drive for metered drug infusion out of a drug reservoir according to a set of infusion regimes. The method includes controlling infusion in a series of scheduled infusion events. The method further includes determining, for an infusion event volume that is infused in a given scheduled infusion event, an assignment between volume fractions of the infusion event volume and infusion regimes in an interlaced manner.

In a further aspect, a computer program product includes a non-transient computer readable medium having stored therein computer program code. The computer program code may be configured to direct a processor to act as infusion device controller and/or to execute a method according to any embodiment as discussed above and/or further below.

The expression “metered drug infusion” refers to a volumetric infusion where the drug volume that is infused as a function of time is controlled, typically by way of positive displacement out of a drug reservoir. The drug is a liquid drug. In an embodiment, in particular in the context of CSII, the drug may be a liquid insulin formulation. Other drugs, such as pain relief drugs or cancer treatment drugs, may be used as well. Infusion is carried out from a common drug source, in particular a common drug reservoir, such as a drug cartridge.

The expression “volume” refers to a liquid drug volume. Volumes may in principle be expressed in any desired physical unit, such as milliliters or microliters. In the context of drug infusion and in particular CSII, however, volumes are frequently expressed in IUs (International Units) of active drug, based on a given concentration, typically U100 (1 milliliter of liquid comprising 100 IUs). Here, 1 IU corresponds to 10 microliters in absolute volume. For liquid drug formulations of other concentration e.g., U40 (1 milliliter of liquid comprising 40 IUs) or U200 (1 milliliter of liquid comprising 200 IUs), an analogues relation applies. Throughout this document, U100 is assumed where not stated differently. Further, volumes may further be expressed as (typically, but not necessarily) multiple of a volume increment.

An infusion regime is defined in its general sense by a volume to be infused as a function of time. As will be discussed in more detail further below, a specific infusion regime may in principle be endless in time, such that infusion is carried out as long as the infusion device is operating. This is particularly the case for a basal regime as discussed further below. Alternatively, a specific infusion regime may be designed for the infusion of a limited and generally pre-determined volume within a limited time period. This is particularly the case for drug boli. The volume of a drug bolus is referred to as bolus volume and total time of the bolus infusion is referred to as bolus duration. Generally, a bolus infusion is, as opposed to basal infusion, an on-demand infusion that may be programmed by a user, for example, by way of direct entry, recalling from a corresponding memory, and/or automatized calculation via a bolus calculator as generally known in the art, at any time.

Generally, the single infusion regimes each define, for a given infusion event an associated infusion volume that shall be infused in a scheduled infusion event. As will become more apparent as the description proceeds, some infusion volumes for some infusion regimes may—and typically are—be zero for a given scheduled infusion event.

Controlling infusion in a series of scheduled infusion events means that infusion event occurs according to a given and generally pre-determined schedule. In an embodiment, the infusion device controller is designed to trigger scheduled infusion events at well-defined points in time, in particular with an equidistant time interval between consecutive scheduled infusion events. For triggering the scheduled infusion events, the infusion device controller may include a clock unit, in particular a real-time clock unit. A typical equidistant time interval is, for example, 3 minutes. Other time intervals such as 1 min, 2 min, 6 min, 10 min or 20 min may be used as well, depending on the overall design and the type of drug. The time interval between scheduled infusion events is referred to as schedule interval. Further, in typical embodiments, the scheduled infusion events are triggered at pre-determined times of day, for example, 0:00 (midnight), 0:03, 0:06, 0:09, 0:12, and so forth or a schedule interval of 3 min. The infusion duration for infusion the scheduled event volume in a scheduled infusion event is generally short as compared to the time interval between the scheduled infusion events and may be in a range of milliseconds up to a number of seconds, depending on the active infusion regimes and the overall design. It is noted that the infusion event volume is generally not constant but varies between the scheduled infusion events.

The infusion event volume is the total volume that is infused in a scheduled infusion event. A volume fraction is a portion (fraction) of a scheduled infusion event that is assigned to a specific infusion regime, with the sum of all volume fractions corresponding to the infusion event volume. The assignment between volume fractions and infusion regimes is unique in that that there is, for each volume fraction, in any case an assignment with exactly one infusion regime. For one and the same infusion regime, however, there may be an assignment with more than one volume fraction. In the context of the present document, the assignment between volume fractions and infusion regimes is generally considered as assigning volume fraction to a given infusion regime. According to an alternative view, however, an infusion regime is assigned to a given volume fraction.

The assignment between volume fractions and infusion regimes being made in an interlaced manner means, for a given scheduled infusion event, the volume is not distributed among the single infusion regimes in a consecutive manner, with one infusion regime following the other. Instead, the total volume that belongs (shall be assigned to) a particular infusion regime is split into volume fractions that alternate with volume fractions belonging (assigned to) other infusion regimes. In some embodiments, the interlaced infusion may in particular be, fully or in part, a cyclic infusion where volume fractions are assigned to two or more infusion regimes in a cyclic manner.

As will be understood as the description proceeds, following this approach avoids a need to delay the infusion according to an infusion regime until infusion according to another infusion regime has been completed. In this way, the computational effort that is required for coordinating and scheduling infusion for the active infusion regimes as well as the complexity of the corresponding algorithm as implemented by the infusion device controller can be significantly reduced.

Further, infusion may be carried out in accordance with a number of infusion regimes in a superimposed way and in a consistent manner for a number of infusion regimes that is in principle unlimited. In accordance with this disclosure, the scheduling of the infusion in accordance with the different infusion regimes and in particular the drive control is simplified in particular for complex scenarios, involving superimposed drug infusion in accordance with several infusion regimes.

For this disclosure, it is important to understand that the assignment between infusion regimes and volume fractions affects the way the infusion device controller operates and further the way how information, in particular real-time information regarding the infusion may be presented to a user. Since all infusion is made from one and the same drug source, the pharmacologic effect, however, is independent from the assignment to the infusion regimes. It is noted that, in dependence of the infusion regimes, a number of volume fractions may be zero.

In an embodiment, the infusion device control unit is designed to assign volume fractions to a number of infusion regimes in a cyclic manner. As will become more readily apparent further below, such embodiment may be implemented in a particular efficient and straight-forward manner.

In an embodiment, the infusion device controller is designed to control, in a given scheduled infusion event, infusion of the infusion event volume as a series of distinct volume packets. After infusion of a volume packet, a pause occurs before the infusion of a next following volume packet. This pause allows an energy supply, typically a rechargeable or non-rechargeable battery, that powers the infusion device to recover. Further, this measure limits the average infusion speed in an often desirable way. As will become more readily apparent further below, a volume packet may directly correspond to a volume fraction in dependence of the implementation and the infusion regimes. Alternatively, however a volume packet may be split into a number of volume fractions that are assigned to different infusion regimes.

In a particular embodiment involving volume packets, the infusion device controller is designed to assign consecutive volume packets to different infusion regimes. For such type of embodiment the total packet volume of a volume packet is assigned to a single infusion regime, thereby forming volume fraction.

In a particular embodiment involving volume packets, the volume packets may have a limited packet volume. A typically pre-determined maximum packet volume accordingly exists that is not exceeded for a single volume packet. Further, in an embodiment, a typically pre-determined packet volume may exist that is not fallen below. A maximum packet volume is favorable for the before-mentioned reasons, in particular to allow battery recovery. A minimum volume packet is favorable regarding the metering precision, since extremely small volumes are critical, if not impossible to meter with the required precision. The infusion device controller may accordingly be designed to activate the drive for the infusion of volume packets between a minimum and/or a maximum volume packet. In a further particular embodiment, the infusion device controller may be designed to activate the drive for the infusion of packet volume of constant packet volume.

In a particular embodiment involving volume packets, the infusion device controller is designed to control, in a given volume packet, infusion as a series of distinct volume increments, and to assign each of the volume increments to an infusion regime. For such embodiment, a volume increment defines the smallest volume that may be infused and assigned an infusion regime. In a typical design of an infusion device, the drive includes a stepper motor and a volume increment corresponds to a step of the stepper motor. The volume increments of a volume packet may all be assigned to the same infusion regime or may be assigned to a number of infusion regimes as explained further below in more detail. Where the volume increments are assigned to a number of infusion regimes, all volume increments that are assigned to the same infusion regime form a volume fraction as explained before. Further, by way of example, a maximum packet volume or a constant packet volume may correspond to 18 volume increments.

In a particular embodiment involving volume packets, the infusion device control unit is designed to assign volume increments of a given volume packet to infusion regimes in accordance with a prioritization order, starting with an infusion regime of highest priority (highest-priority infusion regime), and to assign volume increments to a an infusion regime of lower priority if no further volume increments can be assigned to an infusion regime of higher prioritization order. That no further volume increments can be assigned to an infusion regime of higher priority is the case if a number of volume increments that corresponds to the scheduled infusion volume in accordance with the higher-ranking infusion regime have been assigned to the infusion regime of higher priority. For this type of embodiment, the infusion increments of a volume packet may be assigned block-wise to a number of infusion regimes in accordance with the prioritization order, starting with the highest priority. Where all volume increments are assigned to a single infusion regime (the highest-priority infusion regime), the whole volume packet forms a single volume fraction. Where the volume increments are assigned block-wise to a number of infusion regimes, each of the blocks defines a corresponding volume fraction. In a particular implementation, the prioritization order may be changed in a cyclic way.

In a particular embodiment involving volume packets, the infusion device controller is designed to determine an assignment between the volume packet as a whole to an infusion regime and/or of portions of the volume packet to a number of infusion regimes following the infusion of the volume packet. For this type of embodiment, a volume packet is first infused. Subsequently, its volume is assigned to one or more infusion regimes.

Ideally, the volume that is actually infused, e.g., as infusion event volume, corresponds to the volume for the infusion of which the drive is activated. In reality, however, this can not necessarily be guaranteed for reasons such as (load dependent) step losses, overshooting and undershooting. The effectively infusion volume, e.g., an effective infusion event volume may accordingly deviate from the volume for which the actuator is activated. For embodiments involving the infusion of volume packets as explained before, the effective packet volume that is actually infused may accordingly deviate from a commanded packet volume for the infusion of which the actuator is activated. Similarly, where infusion is carried out as series of infusion increments, the effective (not necessarily integer) increment number may deviate from a commanded increment number and the effective infusion event volume may deviate from the commanded infusion event volume. In an embodiment, determining the assignment between volume fractions and infusion regimes is based on the effective infusion event volume. The infusion device control unit may further be configured to determine effectively infused volumes.

In an embodiment, the set of infusion regimes includes a time-variable and cyclic, in particular circadian, basal regime. In CSII, basal infusion is used to cover the meal-independent insulin demand that is required for maintaining the metabolism. The basal regime is typically pre-programmed. Typically but not necessarily, the basal is circadian, i.e., has a period of 24 hours. In such case that is also assumed in the following for exemplary purposes where not stated differently, the rate of basal infusion is accordingly defined in dependence of the time of day. Typically, a basal infusion profile for the basal regime is stored in form of a basal infusion schedule in a memory of the infusion device controller by way of a lookup table that comprises the total basal infusion volume or the basal infusion rate (volume per time) for time intervals, e.g., for each hour of day or each half hour of day. For each of the time intervals, the basal infusion rate is typically constant, i.e., the basal infusion rate as a function of time is a piecewise constant function. Alternatively, the basal infusion schedule may not be stored in the form of a lookup table, but in the form of a mathematical function respectively the parameters of such function. While basal infusion may be temporarily suspended or modified by a user as explained further below (thereby temporarily deviating from a strictly circadian profile), basal administration is generally carried out by an ambulatory infusion system autonomously under control of an infusion device controller, without requiring user interaction. In typical embodiments, one of the active infusion regimes is always the basal regime.

More than one basal regime may be provided in the set of available infusion regimes for alternative use with either of them generally being an active infusion regime, e.g., for shift works working day shift or night shift, for working days and weekends, or the like. While only one of them is typically active and used at the same time, this disclosure is not limited in this regard and the infusion device controller may also designed to allow superimposed infusion in accordance with more than one basal regime, if desired.

By way of example, it is in the following assumed that the basal infusion as defined by a basal regime is piecewise constant, with basal infusion being defined for each hour of the day. With T as schedule interval of, e.g., 3 min, and V_{Basal_Hour} as basal infusion volume for a given hour of day, the basal event volume V_{Basal} for each scheduled infusion event in this hour of day is accordingly V_{Basal}=V_{Basal_Hour}/(60 min/T). It is noted that the basal event volume V_{Basal} for each scheduled infusion event stays constant over consecutive scheduled infusion events as long as the basal infusion rate is constant.

As mentioned before, while basal infusion is generally cyclic and periodic, this is not necessarily always the case in a strict sense. In particular, the infusion device controller may be designed to allow a user to temporarily modify the basal infusion and raise or lower the basal infusion to cope with special situation such as sportive activities or illness. Further, the infusion device controller may be designed for operatively coupling with a continuous glucose measurement device and to automatically temporarily modify, in particular lower or suspend, basal infusion in dependence of a signal received by the continuous glucose measurement system. Such embodiment is particularly useful to avoid or reduce the occurrence of undesired and potentially dangerous hypoglycaemic episodes in CSII.

In an embodiment, the set of infusion regimes includes an extended bolus regime, extended bolus regime defining a total extended bolus volume to be infused in a limited number of scheduled infusion events, wherein the infusion device controller is designed to receive a request for infusing an extended bolus.

In CSII, extended boli are frequently considered favorable for covering the insulin need for particular types of food that are metabolized by the body over a prolonged time period and may therefore need steady or repeated insulin supply over a period of, e.g., 1 hour, 2 hours, or even longer. The infusion of an extended bolus may be programmed by a device user generally at any time on demand, e.g., by inputting or retrieving from a memory its parameters as explained below, and/or calculating its parameters with a bolus calculator. In some embodiments, more than one extended bolus regime may be independently active at the same time respectively overlap in time. While different types of infusion regimes may be used be used for an extended bolus and an infusion device may be designed for the infusion of different types of extended drug boli, it is here and in the following assumed that an extended bolus is infused in a uniform way. as follows: With V_{Extended_Total} being the total volume of an extended bolus and m being a number of consecutive scheduled infusion events over which its infusion shall be distributed, an extended bolus event volume V_{Extended} for each of the m scheduled infusion events For practical purposes, the duration of the infusion of the extended bolus is entered by a user as absolute time, e.g., as number of hours/minutes, rather than as number of scheduled infusion events.

In an embodiment, set of infusion regimes includes an immediate bolus regime. The infusion device controller is designed to receive a request for infusing an immediate bolus and is further designed to control the drive to start infusion of the immediate bolus asynchronous to the scheduled infusion events.

An immediate bolus is the most commonly used type of drug bolus in CSII and is used for covering the insulin need for metabolizing various types of food, as well as for lowering undesirably raised blood glucose values. The infusion of an immediate bolus may be programmed by a device user generally at any time on demand. An immediate bolus may be defined by its immediate bolus volume as sole parameter. After inputting respectively programming an immediate bolus, in particular its immediate bolus volume, infusion is directly started, independent from the scheduled infusion events. This is favorable because it is normally desirable for an immediate bolus to act as soon as possible. It is noted that, for example, in CSII, bolus volumes may be comparatively large. Typically, the bolus volume is in the range of several IUs and may in some case be up to. e.g., 20 IU or even larger.

In a particular embodiment, the infusion device controller is designed to assign volume fractions to the immediate bolus regime and to further infusion regimes different from the immediate bolus infusion regime in an interlaced manner. Due to its potentially large immediate bolus volume and its asynchronous infusion, infusion of an immediate bolus may timely interfere with a scheduled infusion event, i.e., the infusion of an immediate bolus may be ongoing at the time of a scheduled infusion event. By assigning volume fractions of the scheduled infusion event to immediate bolus regime and further volume fractions to further infusion regimes in an interlaced manner, infusion of the immediate bolus continues during and as part of the scheduled infusion event. Neither needs the infusion of the immediate bolus to be interrupted for the scheduled infusion event, nor needs the infusion according to further infusion regimes to be delayed until infusion of the immediate bolus is completed. After termination of a scheduled infusion event, infusion of the immediate bolus may continue for its yet undelivered portion (i.e., a portion that has neither been infused before nor during and as part of the scheduled infusion event).

In an embodiment, the set of infusion regimes includes a multiwave bolus regime. A multiwave bolus regime comprises, in combination immediate bolus regime and an extended bolus regime.

In CSII, a multi-wave bolus can, similar to an extended bolus as explained before, be used to cover the insulin need for particular types of food. It may in particular be used for food that has a portion that is metabolized by the body over a prolonged time and a further portion that is metabolized within a short time period after intake. The parameters of a multi-wave bolus which are programmed by the user or retrieved from a memory and/or are calculated with a bolus calculator may be its total volume, the immediate portion volume of the portion that is to be infused as immediate bolus, and the time over which infusion of the extended bolus portion is spread. While a dedicated multiwave bolus regime is favorably provided for the infusion of multiwave boli, it may alternatively be programmed as an immediate bolus and a separate extended bolus.

The infusion device controller may in principle be designed to handle any desired number of infusion regimes in parallel. In typical embodiments, the infusion device controller is designed to handle in parallel a basal regime, an immediate bolus regime, one or more extended bolus regimes and one or more multiwave-bolus regimes. Other designs may be used as well. It is further noted that other types of regimes, in particular bolus regimes, may be foreseen as well, for example, a modified extended bolus in which infusion is distributed over an extended time period, corresponding to a number of update periods, with the extended infusion volume varying over time.

In an embodiment, the infusion device controller is designed to provide real-time information with respect to infused drug volumes. Such real-time information may be provided, typically in form of numerical information and/or in graphical form, on a display of the infusion device and/or a remote device in operative coupling with the infusion device. Real-time information may in particular be provided for all types of bolus infusion and may indicate the already administered volume and/or the volume that remains for infusion. It is known that many users, for example, in CSII, tend to manually supervise the infusion of drug boli via the display information. In this context, embodiments in accordance with this disclosure has the particular advantage the actually infused volume and the according real-time information change in an expected and smooth way, without noticeable delays. Delays that are typical to occur for non-interlaced infusion according to the state of the art are known to frequently confuse and irritate a number of users. Real-time information is, in the present context to be understood as information that is continuously updated an reflects the actual state of infusion regarding one or more infusion regimes, in particular already infused bolus volumes and/or remaining bolus volumes to be infused.

In an embodiment, the infusion device controller includes a set of accumulators. Each accumulator is associated with an infusion regime and stores an infusion volume that is scheduled for infusion in accordance with the associated infusion regime. Determining an assignment between a volume fraction and infusion regime involves decreasing the accumulator that is associated with the infusion regime by the volume fraction.

A volume being scheduled for infusion means that it shall be infused by the infusion device without avoidable delay, i.e., generally as soon as possible. The infusion volumes may be stored in the accumulators in different ways, e.g., as absolute volumes or IUs. For the sake of simplicity, it is in the following assumed that they are stored as integer number, namely as multiple of the volume increment. As will become more apparent further below, an accumulator may be considered as account that is increased as volume becomes scheduled for infusion in accordance with an infusion regime and is decreased as volume is respectively has been infused. An infusion volume that is stored by an accumulator as scheduled for infusion may in principle take any value and be in particular larger than a maximum packet volume.

In an embodiment, the infusion device controller is designed to repeatedly execute an infusion control routine. The infusion control routine includes: (a) determining if drug infusion shall be carried out at a current point in time in accordance with the set of infusion regimes. The infusion control routine further includes if it is determined that drug infusion shall be carried out, activating the drive to infuse a volume packet from a drug reservoir as a series of volume increments. The infusion device controller may be further designed to determine an assignment between volume increments of the volume packet and infusion regimes. The volume increments that are assigned to a common infusion regime form a volume fraction as explained before.

The repeated execution of the infusion control routine may be periodic, in particular with a pre-determined period. In a typical embodiment, it is repeatedly carried out in an endless manner for the infusion device being in an operational state. Execution of the infusion control routine at well-defined points in time, in particular with an equidistant time interval as infusion control interval between consecutive executions. The infusion control interval may, e.g., be 1 sec. The infusion control interval is favorably such that infusion according to consecutive infusion control routines is de facto continuous, taking into account the pharmacokinetics of the drug. That is, infusion according to a number of consecutive executions of the infusion control routine may be considered as single infusion over a longer time period. For triggering the executions of the infusion control routine, the infusion device controller may include a clock unit. In a typical example that is also assumed in the following, the infusion control interval is 1 sec. Other infusion control intervals, however, may be used as well. The period is longer as compared to the time that is required for the infusion of a volume packet.

It is noted, however, that an infusion does not necessarily occur for each execution of the infusion control routine. It only occurs if drug infusion shall be carried out at a current point in time in accordance with one or more of the infusion regimes. As will become more apparent further below, actual drug infusion does in fact not occur for a majority of infusion control routines.

For an embodiment that involves repeated execution of an infusion control routine, the accumulators may be updated at respectively shortly before an upcoming scheduled infusion event. In particular, an accumulator that is associated with the basal infusion regime may be set to respectively increased by the basal event volume V_{Basal} to be infused as part of the upcoming scheduled infusion event. An accumulator that is associated with an extended bolus regime may be set the extended bolus event volume V_{Extended} to be infused as part of the upcoming scheduled infusion event. The scheduled infusion event includes a single or number of consecutive executions of the infusion control routine, with one volume packet being infused in each infusion control routine.

For this type of embodiment, an accumulator that is associated with immediate bolus infusion may be increased by respectively set to the immediate bolus volume asynchronous with other accumulators at the time the immediate bolus is programmed respectively its infusion is requested. Infusion of the immediate bolus will then start with the next following execution of the infusion control routine, asynchronous to the scheduled infusion events. If infusion of an immediate bolus interferes with a scheduled infusion event, infusion of the immediate bolus will be continued as part of the scheduled infusion event and in an interlaced manner with further infusion regimes as will become more readily apparent from the description of exemplary embodiments further below.

Further, in a particular embodiment involving the repeated execution of an infusion control routine as explained before, the infusion device control unit may be designed to consider a carry-forward between infusion control routine, the carry forward reflecting a deviation between commanded and effectively infused volumes. This type of embodiment has the advantage that the assignment between infused volume increments on the one side and infusion regimes on the other side is correct and in accordance with the infusion regimes in case of a deviation. On the average and over a number of infusion control routines, the actually infused volumes will match the infusion regimes for this type of embodiment.

Methods for controlling metered drug infusion in accordance with the present disclosure may be carried out using infusion device controllers and infusion devices in accordance with the present disclosure. Therefore, explicitly disclosed embodiments of infusion device controllers and/or infusion devices disclose at the same time corresponding method embodiments and vice versa. In particular, where an infusion device controllers and/or infusion devices may be designed or configured to carry out a particular step or number of steps, a corresponding method embodiment may include such step or number of steps. Similarly, where a method may include a particular step or number of steps, a corresponding infusion device controller or infusion device may be designed or configured to carry out such step or number of steps.

### BRIEF DESCRIPTION OF THE DRAWINGS

The above-mentioned aspects of exemplary embodiments will become more apparent and will be better understood by reference to the following description of the embodiments taken in conjunction with the accompanying drawings, wherein:

FIG. 1 shows an exemplary embodiment of an infusion device along with related elements in a schematic functional view;

FIG. 2 shows a further exemplary embodiment of an infusion device together with a remote device;

FIG. 3 shows the operational flow for the execution of an exemplary infusion control routine;

FIG. 4 shows an exemplary operational flow for determine a volume that is scheduled for infusion the operational flow for the execution of an infusion control routine;

FIG. 5 shows an exemplary operational flow for determine an assignment between volume fractions and infusion regimes;

FIG. 6 shows an exemplary infusion as a function of time;

FIG. 7 shows a detailed view of the diagram shown in FIG. 6;

FIG. 8 shows a further exemplary infusion as a function of time;

FIG. 9 shows a detailed view of the diagram shown in FIG. 8; and

FIG. 10 shows an example for the assignment of volume increments to a number of infusion regimes.

### DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

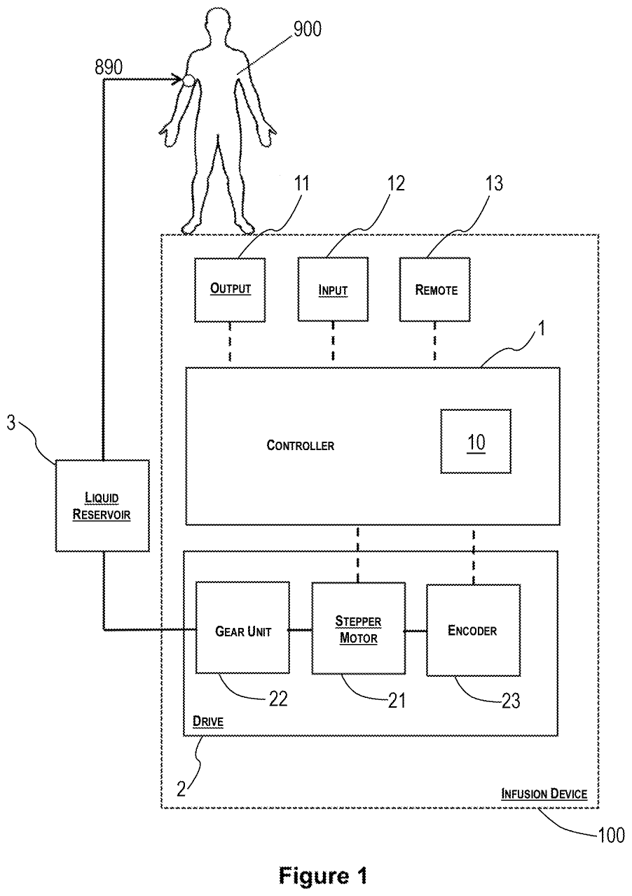

In the following, reference is first made to FIG. 1. FIG. 1 shows an infusion device 100 in accordance with the present disclosure together with associated elements in a schematic functional view. The infusion device 100 includes an infusion device controller 1 and a drive 2. It further includes an output unit 11, input unit 12, and a remote communication unit 13 in operative coupling with the infusion device controller 1.

The infusion device controller 1 is generally realized by electronic circuitry and is typically based on one or more processors, such as microcomputers and/or microcontrollers, and may further includes peripheral components as well as safety circuitry as generally known in the art.

The infusion device 100 further includes a drive 2 that is designed to couple, in an operational configuration, with a drug liquid reservoir 3. The drug reservoir 3 stores a liquid drug for infusion. Here, it is exemplarily assumed that the liquid drug is a liquid insulin formulation of concentration U100 and the infusion device 100 is designed for CSII. Typical reservoir volumes in this application may be in a range of 1 milliliters to 3 milliliters. The drive 2 exemplarily includes a rotatory stepper motor 21 that is powered and thereby activated by infusion device controller 1. The stepper motor 21 that acts on a gear unit 22, and the gear unit 22 acts, in an operational configuration, on a cartridge piston that is sealing and sliding received in a cartridge body of a drug cartridge 3, the drug cartridge 3 forming a drug reservoir. The gear unit 22 may include one or more reduction gears. For transforming the rotatory motor that is generated by the stepper motor 21 into a linear displacement of the cartridge piston, a spindle drive with a threaded spindle is provided. This overall arrangement of the drive 2 forms a so-called syringe driver. An optional rotatory encoder 23 is present that is mechanically coupled with a motor shaft of the stepper motor 21 and further coupled with the infusion device controller 1 for monitoring respectively supervising the infusion.

From the cartridge 3, drug is infused into the body of a user respectively patient 900 via an infusion line 890 in controlled and metered way, in accordance with the displacement of the cartridge piston.

In an embodiment, the infusion device is a compact device with a favorably watertight housing, which is designed to further receive the cartridge 3 in a cartridge compartment. In such embodiment, the housing is favorably sized and shaped to be carried, e.g., with a belt clip or in a pocket. Here, the infusion line 890 typically includes a catheter tubing of, e.g., 0.5 m to 1.5 m length with an infusion cannula. Typically, the infusion device is designed for an application time of several years.

In an alternative design, the infusion device, in particular the control unit 1 and the drive 2, are designed to removable engage a disposable cradle device, the disposable cradle device being designed to be attached to the user's skin via an adhesive pad. Further, cartridge 3 of such embodiment may be designed to reliably engage the infusion device 100 in a side-by-side configuration and to be coupled to the cradle device together with the infusion device 100. Upon coupling, the operatively coupling between the gear unit 22 and the cartridge piston is established. The threaded spindle of the spindle drive may be integral part of the cartridge 3 and the drive nut may be part of the gear unit 22, or vice versa, with a releasable coupling between them. For such embodiment, the infusion line 890 may be reduced to an infusion cannula that directly projects from the skin-contacting surface of the cradle device. In such embodiment, the infusion device 100 may have an application time of several weeks to several years, while the cradle device and the cartridge 3 may have a typical lifetime in a range of a number of days up to, e.g., two weeks. The infusion device 100 is accordingly used with a number of cradle devices and reservoirs in sequence and is accordingly be designed to be attached and detached without damage.

In a further design, the infusion device 100 and the reservoir 3 are designed as a common device that is favorably sealed in a water-tight manner and designed to be directly adhesively attached to the body. For such design, the whole device is designed for a limited application time of a number of days up to a few weeks and to be subsequently discarded.

A power source, typically in form of a rechargeable or non-rechargeable battery may be part of the infusion device 100 or be formed as common unit with the cartridge 3.

The output unit 11 may include a display, for example, a numerical, alphanumerical and/or graphical display. In the context of the present disclosure, it is particularly used to provide real-time information with respect to infused drug volumes as explained above and further below. It may further be used for general user information and/or user feedback purposes as well as programming. The output unit 11 may further include indicators such as a tactile indicator (e.g., a pager vibrator) and/or an acoustic indicator (e.g., a loudspeaker/buzzer) to provide user feedback and for alarming purposes.

The input unit 12 may include one or more pushbuttons and/or other input devices as generally known in the art. The input unit 12 is used for general operation purposes and may in particular be used to program drug boli to be infused, in particular immediate boli.

The optional remote communication unit 13 is provided for communication with a further device, in particular a remote controller (not shown). Such remote controller may be a dedicated device or may be a general-purpose device, such as a Smartphone, running a corresponding application. In addition to other functionalities such as general therapy tracking and monitoring, a further device may be used for programming infusion in accordance with one or more infusion regimes, alternatively or additionally, however, infusion regimes basal infusion profiles, extended bolus infusion profiles and a multi-wave bolus may be programmed using the input unit 12 and the output unit 11. It is noted that the programming an infusion regime is generally meant as programming its parameters.

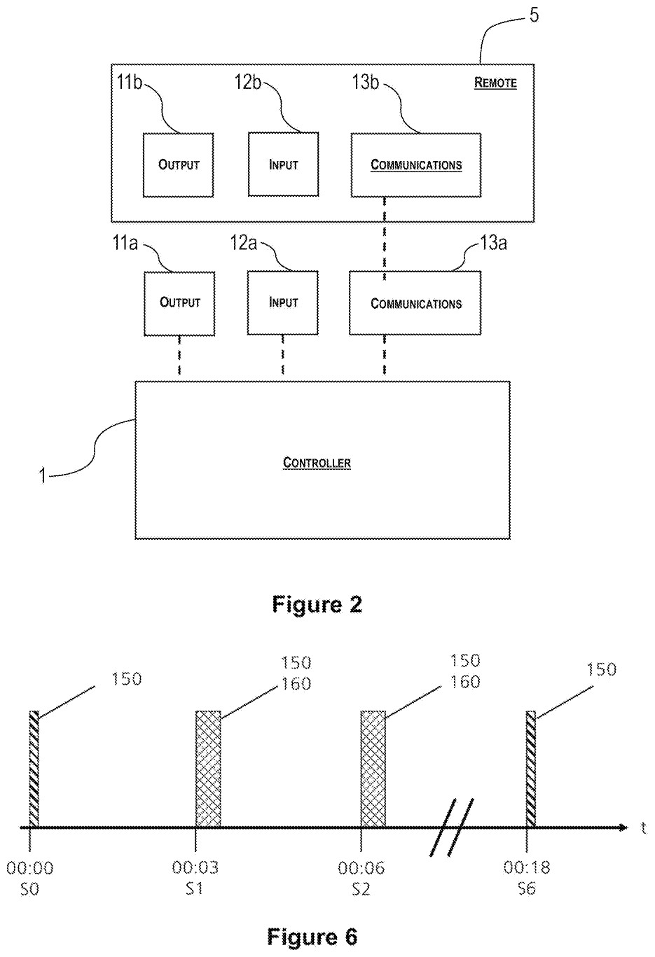

In the following, reference is additionally made to FIG. 2, showing a further embodiment of an infusion device controller 1 together with a remote device 5 in a schematic functional view. The remote device control unit 1 of this embodiment differs from the embodiment in FIG. 1 in particular in that it comprises an output unit 11a and an input unit 12a of limited functionality. In this embodiment, the output unit 11a is a tactical and/or acoustic indication unit which serves mainly for alarming purposes. The input unit 12a is a pushbutton and exemplarily used only for programming immediate boli with a given bolus volume increment of, e.g., 0.5 IU per actuation. A remote device 5 is provided with a remote device output unit 11b, a remote device input unit 12b, and a remote infusion device communication unit 13b in operative coupling with the communication unit 13a of the infusion device 100 via a communication link, typically an RF communication link. The remote device input unit 12b and the remote device output unit 11b are, in this embodiment, generally designed for operating and programming the infusion device 100. They are in particular designed for programming parameters of infusion regimes, e.g., the volume of an immediate bolus or the infusion volume for each hour of a day for a basal regime.

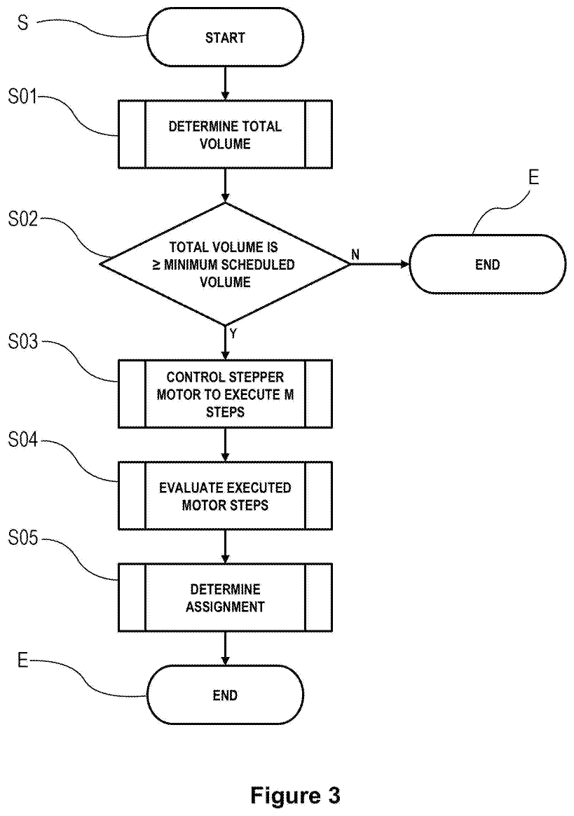

In the following, reference is additionally made to FIGS. 3 to 5. FIG. 3 illustrates on a high level the operational flow of an infusion control routine that is executed under control of the infusion device controller 1 with an infusion control interval of exemplary 1 sec, starting with step S.

The infusion device controller 1 comprises a set of accumulators, wherein each accumulator as explained above in the general description. Each accumulator is associated with an infusion regime. For merely exemplary purposes, the following five infusion regimes are assumed to be available:

A immediate bolus

B multiwave bolus (immediate bolus portion)

C multiwave bolus (extended bolus portion)

D extended bolus

E basal infusion

As explained in the general description before, the extended bolus portion and the immediate bolus portion of an extended portion are favorably programmed respectively inputted together.

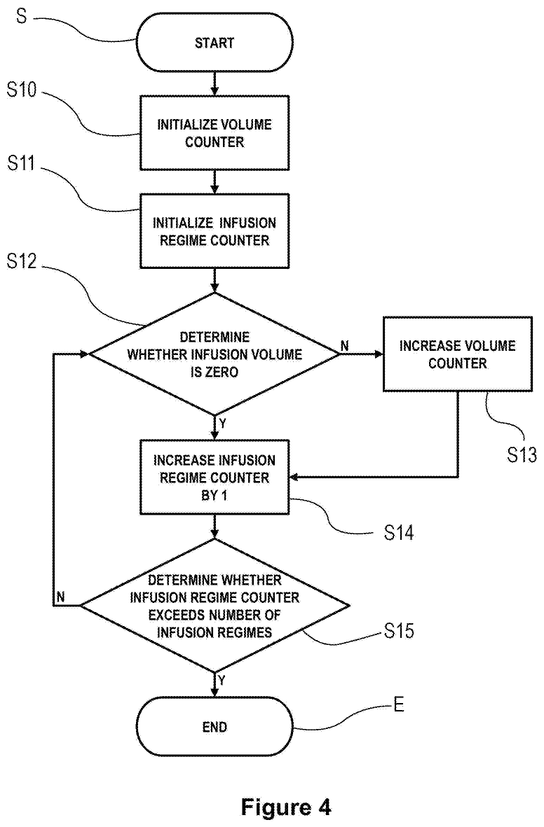

In step S01, the total volume that is scheduled for infusion at the present point in time is determined. This total volume is determined by the sum of volumes that are stored by the accumulators for the exemplary infusion regimes A, B, C, D, E and as explained in more detail further below in the context of FIG. 4.

In optional step S02, it is determined whether the total volume that is scheduled for infusion is at least a minimum scheduled volume, that may correspond to a minimum number of, e.g., M=18 motor steps of the stepper motor 21. As explained further below, this corresponds to the regular packet volume. If this is not the case, the operational flow ends (step E) and is executed again with the next infusion control routine after expiry of the infusion control interval. In this case, no infusion is carried out in the present infusion control interval. In a variant, the minimum number of motor steps (and accordingly the minimum packet volume) may be smaller, for example, M′=4 motor steps for the last volume fraction of an immediate bolus or extended bolus. Thereby, it is ensured that the full bolus volume is infused without delaying it to the next scheduled infusion event.

If at least the minimum number of steps is scheduled for infusion, the operational flow proceeds with step S03 where the infusion device control unit controls the stepper motor 21 to execute the M steps, thereby infusing a volume packet, with each motor step corresponding to a volume increment.

In subsequent step S04, the number of actually executed motor steps is evaluated by way of evaluating a signal that is provided by the rotatory encoder 23 during actuation of the stepper motor 21. It is noted that, in dependence of the overall design of the ambulatory infusion device 100 and in particular the drive unit 2 as well as the way it is controlled, the number of motor steps as actually carried out by the stepper motor 21 may not exactly correspond to the commanded step number M that it has been actuated to execute. That is, an effective step number M_{effective} respectively effective volume increment number may deviate from the commanded step number M as explained above in the general description, resulting in the packet volume as infused in step S03. The effective packet volume (determined by the number of actually executed motor steps) may accordingly deviate from the commanded packet volume.

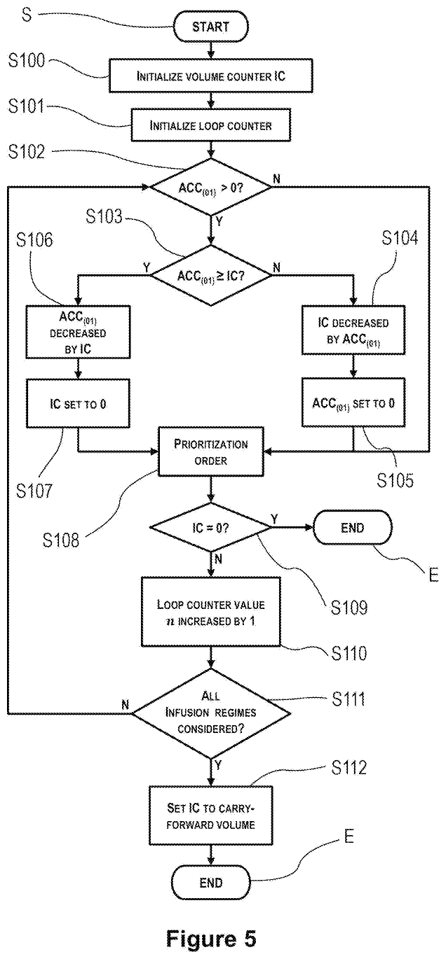

In subsequent step S05, an assignment is determined between the effectively executed motor steps M_{effective} respectively the associated effectively infused volume increments and the infusion regimes, as explained in more detail further below in the context of FIG. 5. The operational flow ends (step E) and is executed again with the next infusion control routine after expiry of the infusion control interval. While being favorable, it is noted that a distinction between the commanded step number and effective step number and associated volume increments and packet volume is not absolutely mandatory, if it can be assured by other measures that the commanded step number is effectively executed.

FIG. 4 illustrates in more detail the operational flow for determine the total volume that is scheduled for infusion in an execution of the infusion control routine (S01 in FIG. 3).

The operational flow starts with step S. In subsequent step S10, a scheduled volume counter is initialized with V_{Scheduled}=0. In subsequent step S11, an infusion regime counter is initialized with n=1, indicating a first of the infusion regimes. In subsequent step 12, it is determined whether the infusion volume that is stored in the accumulator as indicated by the value of the infusion regime counter n is zero and the operational flow branches in dependence of the result. In the negative case, the operational flow proceeds with step S13 where the scheduled volume counter V_{Scheduled} is increased by the infusion volume as stored in the accumulator and the operational flow proceeds with step S14. In the affirmative case of step S12 (infusion volume is zero), step S13 is skipped and the operational flow proceeds with step S14 directly after step S12.

In step S14, the infusion regime counter n is increased by 1 for considering a next infusion regime. In subsequent step S15, it is determined if the infusion regime counter n exceeds the number of infusion regimes. In the affirmative case, the operational flow ends with step E. In the negative case, the operational flow returns to step S12 which is accordingly carried out for the next infusion regime. Since all infusion regimes are considered in this way, the order of the infusion regimes is not decisive.

FIG. 5 illustrates in more detail the operational flow for determining the assignment between the effectively executed motor steps respectively the associated effectively infused volume increments M_{effective} and the infusion regimes (S05 in FIG. 3).

The operational flow starts with step S. In subsequent step S100, a volume counter IC is initialized with the effectively infused packet volume, as determined from the effective step number M_{effective} in step S04, plus a (positive or negative) carry-forward volume, if applicable. As explained in more detail in the following, the volume counter IC indicates the number of volume increments that where infused in step S04 and have not yet been associated with an infusion regime. In subsequent step S101, a loop counter is initialized with n=1. It is noted that the effectively infused packet volume is directly given by the effective step number M_{effective} if volumes are considered as multiples of the volume increment.

In subsequent step S102, it is determined whether an accumulator ACC₍₀₁₎ that is associated with the priority infusion regime (see step S108 below) stores a value that is positive respectively different from zero, in which case volume increments are to be assigned to the priority infusion regime. If this is not the case, i.e., if no volume increments are to be assigned to the priority infusion regime, the operational flow proceeds with step S108 as explained further below.

If it is determined in step S102 that the accumulator ACC₍₀₁₎ is positive, is positive, the operational flow proceeds with step S103. In this case, at least one infused volume increment has not yet been assigned with an infusion regime. In step S103, the volume that is stored in an accumulator ACC₍₀₁₎ is compared with the value IC of the volume counter, and the operational flow proceeds in dependence of the result. If the volume ACC₍₀₁₎ is at least the value IC of the volume counter, the operational flow proceeds with step S106. In step S106, the volume ACC₍₀₁₎ is decreased by the value IC of the volume counter, i.e., ACC₍₀₁₎=ACC₍₀₁₎−IC. In this step S106, all remaining volume increments of the volume packet are assigned with the highest-priority infusion regime. Consequently, the total volume of the volume increments as assigned in step S06 form a common volume fraction. Since no further volume increments of the current volume packet are open for assignment with an infusion regime, the value IC of the volume counter is set to zero in subsequent step S107 and the operational flow proceeds with step S108. As will become more readily apparent further below, the volume ACC₍₀₁₎ may be identical to or exceed the effective packet volume. In this case, all volume increments are assigned this highest-priority infusion regime as single volume fraction.

If it is determined in step S103 that the volume as stored in the highest-priority accumulator ACC₍₀₁₎ is smaller than the value IC of the volume counter, the operational flow proceeds with step S104. In step S104, the value IC of the volume counter is decreased by the volume ACC₍₀₁₎. In this step, a number of volume increments that corresponds to the volume ACC₍₀₁₎ is assigned with the highest-priority infusion regime. Since no further volume is open for infusion in accordance with the highest-priority infusion regime, the value of the corresponding accumulator is set to zero, ACC₍₀₁₎=0, in subsequent step S105 and the operational flow proceeds with step S108. The total volume of the volume increments that are assigned in step S104 form a common volume fraction, which, however, is less than the packet volume in this case.

In subsequent step S108, the prioritization order is modified as explained in the following. The prioritization order indicates the order in which volume increments are assigned to the infusion regimes in step S104 or S106. By way of example, two prioritization orders are shown in Table 1-1, 1.2.

TABLE 1-1

| ↓ | | | | |
|---|---|---|---|---|
| (01) | (02) | (03) | (04) | (05) |
| A | B | C | D | E |

TABLE 1-2

| ↓ | | | | |
|---|---|---|---|---|
| (01) | (02) | (03) | (04) | (05) |
| B | C | D | E | A |

In Tables 1-1, 1-2, (01), (02), . . . indicate prioritization orders, showing an exemplary prioritization order before (Table 1-1) and after (Table 1-2). Prioritization order (01) is indicated by an arrow and defines the priority infusion regime. The volume increments are assigned in step S014 respectively S106 to this priority infusion regime.

Exemplarily assuming that a prioritization order according Table 1-1 applied for an execution of steps S102 . . . S107 as explained before, volume increments where accordingly assigned to the immediate bolus regime A as highest-priority infusion regime.

In step S108, the prioritization order is changed to a prioritization order according to Table 1-02 via a cyclic rotation by one position. Consequently, a multiwave bolus (immediate bolus portion) B is now the highest-priority infusion regime. With the next executions of steps S102 . . . S107 as explained below, volume increments will accordingly be assigned to multiwave bolus (immediate bolus portion) B. With each execution of step S108, a cyclic rotation by one position is carried out.

In subsequent step S109, the value IC of the volume counter is compared against zero and the operational flow branches in dependence of the result. If the value IC of the volume counter is zero, all volume increments of the present infusion package have been assigned to an infusion regime and the operational flow ends with step E.

If the value IC of the volume counter is different from zero, not all volume increments have been assigned to an infusion regime and the operational flow proceeds to step S110 where the value n of the loop counter is increased by one.

In subsequent step S111, the value n of the loop counter is compared against the number of infusion regimes, e.g., 5 in this example. It is determined that the value n of the loop counter is not bigger than the number of infusion regimes, not all of the infusion regimes have been considered for assigning volume increments. In this case, the operational flow returns to Step S012. The infusion regime to which volume increments may be assigned, however, has changed because of the modified prioritization order in step S108. It is noted that, due to step S102, no volume increments are assigned with the highest-priority infusion regime if the corresponding accumulator stores a value of zero, effectively resulting in this infusion regime being skipped in the assignment between volume increments and infusion regimes.

If it is determined in step S111 that all infusion regimes have been considered, the operational flow proceeds with step S112. In step S112, the volume counter IC is set to the carry-forward volume. The carry-forward volume is the deviation between the commanded packet volume and the effectively infused packet volume. The operational flow terminates with step E.

Regarding the operational flow according to FIG. 5, it is further noted that step S112 only occur in cases where a deviation is present between the commanded and effectively infused packet volume respectively between the commanded step number and effectively executed step number of the stepper motor 21. If both math, the operational flow will terminate after step S109.

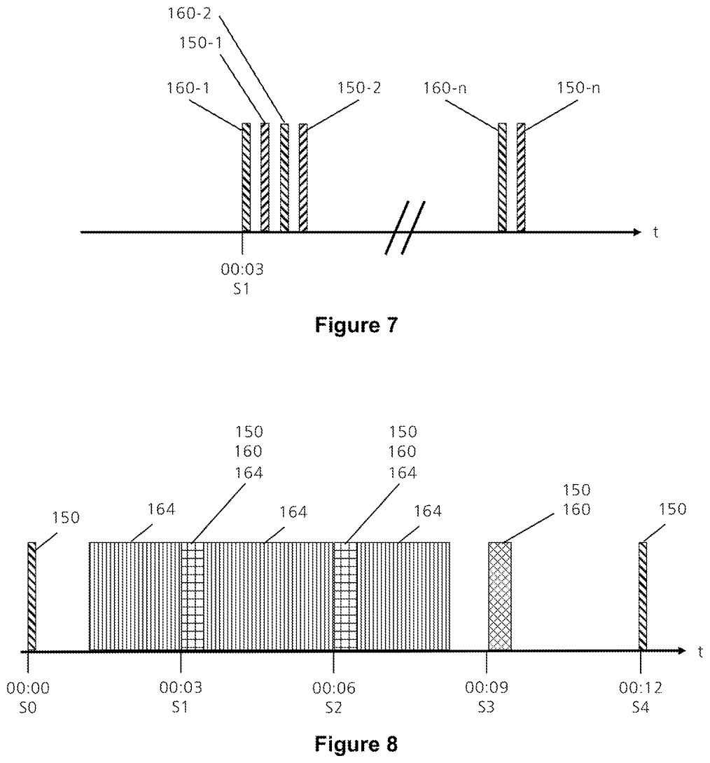

In the following, reference is additionally made to FIGS. 6, 7, illustrating the infusion according to a basal regime and an additional extended bolus regime. Reference is first made to FIG. 6. Like in further following examples, the shown time axis exemplarily starts at 00:00 (midnight). It is further assumed that accumulators may be generally set respectively increased in accordance with one or more infusion regimes with a schedule interval of T=3 min (except for an immediate bolus as discussed further below) and the infusion control routine is executed periodically with an infusion control interval of 1 sec. This results in a scheduled infusion event with the infusion of one or more volume packets every 3 min (with a scheduled infusion event being a consecutive infusion of one or more volume packets in a corresponding number of executions of the infusion control routine). The scheduled infusion events are indicated as Sx, with x being a running number.

Further, are shown as bars, with the width (extension in t-direction) indicating the volume that is infused, corresponding to a number of volume packets respectively volume increments. Each volume increment is associated with a step that is executed by the stepper motor 21. It is noted the shown widths of the bars does not show the actual duration of an infusion, but is exaggerated. It is further assumed that at the beginning all accumulators store a value of zero, indicating that no infusion is scheduled.

At the scheduled event S0 (0:00 (midnight)), the value of an accumulator ACC_{Basal} that is associated with the basal regime is increased by a basal event volume V_{Basal} to be infused, for example, 1/20th of the total basal infusion volume V_{Basal_Hour} that is programmed for the period between 0:00 (midnight) and 01:00. At the following execution of the infusion control routine, the total volume that is scheduled for infusion (step S01 in FIG. 3; 4) is set to the value stored by the accumulator ACC_{Basal}. Since all other accumulators store a value of zero, the other infusion regimes do at this point not contribute to the total volume. The stepper motor 21 is actuated for the infusion of a volume packet (step S05 in FIG. 3). By way of example, it is assumed that the basal event volume V_{Basal} corresponds to a number of n volume packets of the maximum packet volume, with the maximum packet volume corresponding to a maximum number of exemplary 18 volume increments respectively motor steps.

When determining the assignment between volume increments and infusion regimes (FIG. 5), all volume increments of the infused volume packet will be assigned to the basal regime and the accumulator ACC_{Basal} decreased accordingly. After the execution of the infusion control routine, the accumulator ACC_{Basal} will store a volume that corresponds to the n−1 fold of the maximum packet volume. Therefore, in the next following executions of the infusion control routine (i.e., for each of the next following seconds in accordance with the infusion control interval of 1 sec.), the before-mentioned steps will be repeated, until the full basal event volume V_{Basal} has been infused and the accumulator ACC_{Basal} again stores a value of zero (not considering carry-forwards for the sake of clarity). The basal infusion is accordingly distributed over a number of n consecutive executions of the infusion control routine. In FIG. 6, the total basal infusion is shown as infusion event 150. It is further noted the occurring modifications of the prioritization order do not have any effect so far as only basal infusion is carried out. It is further noted, that in the operational flow of FIG. 5 only the branch of steps S106, S107 is used since in each execution of the infusion control routine all volume increments are assigned to the basal regime (resulting the basal event volume V_{Basal} being an integer multiple of the maximum packet volume in this example).

After n consecutive executions of the infusion control routine, the accumulator ACC_{Basal} store a value of zero, indicating, that no further basal infusion is scheduled. Since all other accumulators also store a value of zero, no infusion is carried out in subsequent executions of the infusion control routine.

Further, in the example as illustrated in FIGS. 6, 7, an extended bolus with a total extended bolus volume V_{Extended_Total} is programmed at some point in time after the first scheduled infusion event S0, but before the next following scheduled infusion event S1. Exemplarily, the extended bolus of volume is programmed to have a duration of 15 minutes. An accumulator ACC_{Extended} that is associated with the extended bolus regime will accordingly be set for m=5 consecutive scheduled infusion events extended infusion volume V_{Extended}=V_{Extended_Total}/m.

For each of the following infusion evens S1, S2, S3, S4, S5, both the accumulator ACC_{Basal} that is associated with the basal regime and the accumulator ACC_{Extended} that is associated with the extended infusion regime will accordingly have a value that is different from zero. For each of S1, S2, S3, S4, S5, a basal infusion 150 and an extended bolus infusion 160 will occur in combination and in an interlaced manner as further explained below in the context of FIG. 7. It is noted that after the completion of each scheduled infusion event, both accumulators ACC_{Basal} and ACC_{Extended} store a value of zero and are set again to a value different from zero at the next following scheduled infusion event. For clarity reasons, only the scheduled infusion events S1, S2 are shown in FIG. 6. Scheduled infusion events S3, S4, S5 (at 00:09, 00:12, 00:15) are carried out the same way. With the scheduled infusion event at S5, infusion of the extended bolus is complete and a pure basal infusion 150 again occurs for S6, like for S0 before.

FIG. 7 shows a detailed view for the infusion event at 00:03 (S1). For the sake of clarity, it is here first assumed that the extended infusion volume V_{Extended} for each of the scheduled infusion event is the same as the basal event volume V_{Basal} for each of the scheduled infusion events. In FIG. 7, the individual volume packets are referred to as 150-y (if belonging to the basal infusion) or 160-y (if belonging to the extended bolus infusion), with y being a running number.

Exemplarily, it is further assumed that a prioritization order according to Table 1-1 applies at the beginning of the first execution of the infusion control routine. Since, however the accumulators that are associated with the infusion regimes A (immediate bolus), B (multiwave bolus, immediate bolus portion, C (multiwave bolus, extended bolus portion) exemplarily each store a value of zero, no volume increments are assigned to these infusion regimes, but a sequence S102→S108→S109→S110→S111 is repeatedly run through. In step S108, the prioritization order is modified each time by way of cyclic rotation, thereby changing the highest-priority infusion regime, until finally a prioritization order according to Table 1-3 applies:

TABLE 1-3

| ↓ | | | | |
|---|---|---|---|---|
| (01) | (02) | (03) | (04) | (05) |
| D | E | A | B | C |

Therefore, the volume increments of the volume packet 160-1 that is infused in this execution of the infusion control routine are assigned to the extended bolus regime D. With execution of step S108, the prioritization order is modified as shown in Table 1-4, making basal regime E the highest-priority infusion regime.

TABLE 1-4

| ↓ | | | | |
|---|---|---|---|---|
| (01) | (02) | (03) | (04) | (05) |
| E | A | B | C | D |

Consequently, the volume increments of the volume packet 150-1 that is infused with the next following execution of the infusion control routine volume are assigned to the basal regime E.

The here-described mechanism of alternative assignment of volume packets to the extended bolus regime D and the basal regime E is repeated for the next following executions of the infusion control routine with the infusion of volume packages 160-2, 1502, . . . 160-n, 150-n. After the infusion of volume package 150-n and corresponding assignment to the basal regime E, both involved accumulators ACC_{Extended} and ACC_{Basal} again store a value of zero, and no volume packet is accordingly infused in the next following executions of the infusion control routine, until the accumulators are updated again at S2. The volume packets 160-1, 150-1, . . . 160-n, 150-2 form, in combination, the infusion event volume, with the volume packets each forming a volume fraction. In this example, volume fractions directly correspond to volume packets.

In a variant that is more typical from a practical point of view, the extended infusion volume V_{Extended} for each of the scheduled infusion events is larger as compared to the basal event volume V_{Basal}. In such scenario, the before-described procedure would be followed in the same way. Following the last basal infusion 150-n, however, the accumulator ACC_{Extended} for the extended infusion regime would still store a value different from zero in accordance with remaining volume portion of the extended bolus. Infusion of the extended bolus only would in this situation continue with following executions of the infusion control-routine.

It is noted that the specific prioritization order at the beginning of the infusion event is not deceive and dependent on the history of past infusions.

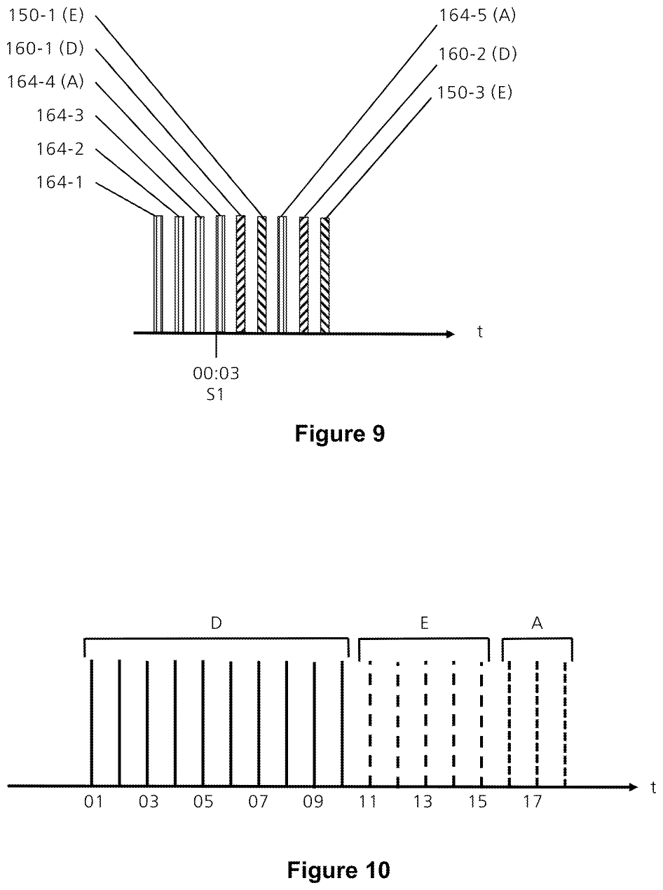

In the following reference is additionally made to FIGS. 8, 9. FIGS. 8, 9 largely correspond to FIGS. 6, 7 for an exemplary scenario that involves basal infusion, extended bolus infusion, and additional infusion of an immediate bolus.

At the beginning, the situation corresponds to the before-described example, and basal infusion 150 is accordingly carried out in the first scheduled infusion event S0.

At some points between S0 and S1, both an extended bolus with a total extended bolus volume V_{Extended_Total} and an additional immediate bolus with an immediate bolus volume V_{Immediate} are programmed. Exemplarily, the extended bolus of volume is programmed to have a total duration of 9 minutes only in this example and is accordingly infused over 3 consecutive scheduled infusion events.

As explained before in the general description, the accumulator ACC_{Immediate} is updated respectively immediately when programming the immediate bolus to the immediate bolus volume, ACC_{Immediate}=V_{Immediate}, asynchronous, with scheduled infusion events. Therefore, infusion of the immediate bolus 164 is started substantially immediate with the next execution of the infusion control routine. Since the accumulators that are associated with the other infusion regimes store a value of zero, no volume packets respectively volume increments are assigned to these infusion regimes at the beginning.

In this example, the immediate bolus volume V_{Immediate} is sufficiently large not to be fully infused at the following scheduled infusion event time S1, thereby causing an interference. Here, the accumulators ACC_{Basal} and ACC_{Extended} for the basal regime and extended bolus regime are updated, ACC_{Basal}=V_{Basal} and ACC_{Extended}=V_{Extended} as explained before. In following executions of the infusion control routine, combined basal infusion 150 and infusion of the extended bolus 160 and the immediate bolus 164 is carried out, as explained in more detail with reference to FIG. 9. Subsequently, infusion of the immediate bolus 164 is continued (between S1 and S2) because the accumulators ACC_{Immediate1} still stores a value different from zero. It is further assumed that the immediate bolus volume V_{Immediate} has not been fully infused at the following scheduled infusion event S2. Therefore, combined infusion will occur in the same way as for S1. Subsequently, infusion of the immediate bolus 164 continues until it is completed. With the next following scheduled infusion event S3, combined infusion of the of the extended bolus and basal infusion occur in the same way as explained before with reference to FIGS. 6, 7.

FIG. 9 shows, similar to FIG. 7, a detailed view for the scheduled infusion event S1 (together a history of three preceding immediate volume packets for the immediate bolus infusion, 164-1, 164-2, 164-3. Exemplarily, it is again assumed that a prioritization order according to Table 1-1 applies at the beginning of the first execution of the infusion control routine for S1. Consequently, the volume packet in the first execution of the infusion control routine is assigned to the immediate bolus regime A. In accordance with the before-explained modification of the prioritization order by way of cyclic rotation, the next following volume packet 160-4 will be assigned to the extended bolus regime D, followed by a volume packet 150-1 that is assigned to the basal infusion regime E, and so forth.

It can be seen that the volume packets are assigned to the immediate bolus regime A, the extended bolus regime D and the basal regime E, in an interlaced and alternating manner, with each of the volume packets defining a volume fraction. Here, the infusion event volume corresponds to the total volume that is infused as part of a scheduled infusion event and ends with the last execution of the drive control sequence where basal infusion or extended bolus infusion is carried out. The subsequent continuation of the immediate bolus infusion may be considered as separate from the scheduled infusion event.

The output unit 11 may provide real-time information with respect to infused drug volumes, in particular for the extended bolus and the immediate bolus in a smooth manner since the volume packets are distributed among the infusion regimes. Further, no particular algorithm is required for delaying infusion according to any infusion regime, since all infusion regimes are treated in the same way and in a consistent manner. Particularly, the infusion of the immediate bolus does neither substantially delay the infusion according to the other infusion regimes at the update points in time, nor is interrupted or substantially delayed.

In the following, reference is additionally made to FIG. 10, illustrating how single motor steps of the stepper motor 21 of the infusion of a volume packet may be assigned to different infusion regimes in an infusion control routine according to a further example. Unlike the before-discussed examples, the accumulators ACC_{Basal}, ACC_{Extended} and ACC_{Immediate} each store a value that is smaller than the maximum packet volume. Exemplary the accumulator ACC_{Basal} stores a volume that corresponds to 5 volume increments, the accumulator ACC_{Extended} stores a volume that corresponds to 10 volume increments, and the accumulator ACC_{Immediate} stores a volume that corresponds to at least 3 volume increments. It is further exemplarily assumed that Table 1-3 applies for the prioritization order at the beginning. Following the activation of the stepper motor for the infusion of a volume packet having the maximum packet volume with M=18 motor steps, the motor steps are assigned to the infusion regimes as follows: First, 10 motor steps 01-10 are assigned to the extended infusion regime D, resulting in the associated accumulator ACC_{Extended} storing a value of zero. Following a cyclic modification of the prioritization order, 5 volume increments 11-15 are assigned to the extended infusion regime E, resulting in accumulator ACC_{Extended} storing a value of zero. Following a further cyclic modification of the prioritization order, the remaining 3 volume increments 15-18 are assigned to the immediate bolus regime A. It is noted that the accumulator ACC_{Immediate} does not necessarily store a value of zero at the end. But infusion of the immediate bolus may be continued with further executions of the infusion control routine. In this example, the volume increments that are assigned to the same infusion regime (e.g., 01-10; 11-15; 16-18) each define a volume fraction.

While exemplary embodiments have been disclosed hereinabove, the present invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

What is claimed is:

1. An infusion device controller for controlling a drive for metered drug infusion from a drug reservoir according to a set of infusion regimes, including an immediate bolus regime, wherein the infusion device controller is configured to:
control infusion in a series of scheduled infusion events;
receive a request for infusing an immediate bolus and to control the drive to start infusion of the immediate bolus asynchronous to the scheduled infusion events;
determine, for an infusion event volume that is infused in a given scheduled infusion event, an assignment between volume fractions of the infusion event volume to the immediate bolus regime and to further infusion regimes different from the immediate bolus infusion regime in an interlaced manner; and
control, in a given scheduled infusion event, infusion of the infusion event volume as a series of distinct volume packets.

2. The infusion device controller according to claim 1, wherein the infusion device controller is configured to trigger scheduled infusion events at defined points in time.

3. The infusion device controller according to claim 2, wherein the infusion device controller is configured to trigger scheduled infusion events with an equidistant time interval between consecutive scheduled infusion events.

4. The infusion device controller according to claim 1, wherein the infusion device controller is configured to assign volume fractions to a number of infusion regimes in a cyclic manner.

5. The infusion device controller according to claim 1, wherein the infusion device controller is configured to assign consecutive volume packets to different infusion regimes.

6. The infusion device controller according to claim 5, wherein the volume packets have a limited packet volume.

7. The infusion device controller according to claim 5, wherein the infusion device controller is configured to control, in a given volume packet, infusion as a series of distinct volume increments and to assign each of the volume increments to an infusion regime.

8. The infusion device controller according to claim 7, wherein the infusion device controller is configured to assign volume increments of a given volume packet to infusion regimes in accordance with a prioritization order, starting with an infusion regime of highest priority, and to assign volume increments to a an infusion regime of lower priority if no further volume increments can be assigned to an infusion regime of higher prioritization order.

9. The infusion device controller according to claim 1, wherein the set of infusion regimes includes a time-variable and a cyclic basal regime.

10. The infusion device controller according to claim 9, wherein the basal regime is circadian.

11. The infusion device controller according to claim 1, wherein the set of infusion regimes includes an extended bolus regime, the extended bolus regime defining a total extended bolus volume to be infused in a limited number of scheduled infusion events, wherein the infusion device controller is configured to receive a request for infusing an extended bolus.

12. An infusion device controller according to claim 1, wherein the infusion device controller has a set of accumulators, wherein each accumulator is associated with an infusion regime and stores an infusion volume that is scheduled for infusion in accordance with the associated infusion regime, wherein determining an assignment between a volume fraction and infusion regime involves decreasing the accumulator that is associated with the infusion regime by the volume fraction.

13. An infusion device controller according to claim 1, wherein the infusion device controller is configured to provide real-time information with respect to infused drug volumes.

14. An infusion device for drug infusion for an extended time period and adapted to be carried extracorporally by a user for the extended time period and concealed from view, the infusion device comprising:
an infusion device controller according to claim 1; and
a drive operatively coupled with the infusion device controller, the drive being configured to couple to a drug reservoir for infusing drug out of the drug reservoir.

15. A method for controlling a drive for metered drug infusion from a drug reservoir according to a set of infusion regimes, including an immediate bolus regime, the method comprising:
controlling infusion in a series of scheduled infusion events;
receiving a request for infusing an immediate bolus asynchronous to the scheduled infusion events;
determining, for an infusion event volume that is infused in a given scheduled infusion event, an assignment between volume fractions of the infusion event volume to the immediate bolus regime and to further infusion regimes different from the immediate bolus infusion regime in an interlaced manner; and
in a given scheduled infusion event, controlling infusion of the infusion event volume as a series of distinct volume packets.

16. The method of claim 15, further comprising scheduling infusion events at equidistant time intervals between consecutive scheduled infusion events.

17. The method of claim 15, further comprising assigning volume fractions to a number of infusion regimes in a cyclic manner.

18. The method of claim 15, further comprising assigning consecutive volume packets to different infusion regimes.

19. The method of claim 18, wherein the volume packets have a limited packet volume.

20. The method of claim 19, further comprising, in a given volume packet, infusing as a series of distinct volume increments and assigning each of the volume increments to an infusion regime.
